(19) United States
(12) Patent Application Publication
(10) Pub. No.: US 2025/0375226 A1
(43) Pub. Date: Dec. 11, 2025

(54) Dynamic Axial Nail For Intramedullary Treatment Of Long Bone Fractures

(71) Applicant: Satori Orthopaedics, Inc., Phoenix, AZ (US)

(72) Inventor: Navid Ziran, Phoenix, AZ (US)

(73) Assignee: Satori Orthopaedics, Inc., Phoenix, AZ (US)

(21) Appl. No.: 19/229,990

(22) Filed: Jun. 5, 2025

Related U.S. Application Data
(60) Provisional application No. 63/656,710, filed on Jun. 6, 2024.

(51) Int. Cl. A61B 17/72; A61B 17/00

(52) U.S. Cl. CPC A61B 17/72; A61B 2017/00477

(57) ABSTRACT

An intramedullary nail includes a proximal body portion and a distal body portion coupled to each other for axial reciprocation. The proximal and distal body portions define a length of the intramedullary nail extending between opposed proximal and distal ends of the intramedullary nail. An inflection point is located along the length, where the length is bifurcated into a proximal length proximal to the inflection point and a distal length distal to the inflection point. Proximal bores are located proximate the proximal end of the intramedullary nail. Distal bores are located proximate the distal end of the intramedullary nail. An interlocking screw hole is located proximate to and distal from the inflection point. The interlocking screw hole is located along the distal length in the proximal body portion of the intramedullary nail.

### CROSS-REFERENCE TO RELATED APPLICATIONS

This application claims the benefit of U.S. Provisional Application No. 63/656,710, filed Jun. 6, 2024, which is hereby incorporated by reference in its entirety.

### FIELD

The present specification relates generally to medical devices, and more particularly to orthopaedic medical devices.

### BACKGROUND

When a bone breaks or fractures, there is a crucial need to set it in the correct position and stabilize it until it heals. Failure to properly stabilize a fracture can result in deformity and/or a painful, non-healing fracture. Healthcare providers typically manage fractures through either non-surgical or surgical treatments. Non-surgical treatment usually involves splinting, while surgical treatment entails securing the fracture with implants such as plates and screws or, in the case of a long bone (such as the femur, tibia, humerus, etc.), an intramedullary nail or rod inserted into the bone's canal.

An intramedullary nail functions as an internal splint and is load-sharing. It is stabilized to the bone with interlocking screws placed above and below the fracture, and it distributes load between the bone and the implant. This load-sharing characteristic of intramedullary nails promotes mechanotransduction, a process critical for bone healing at the molecular level.

The first intramedullary nails were made of stainless steel, which is significantly stiffer than bone. These steel implants bore most of the load placed on a bone, reducing the mechanical stimulus needed for bone healing, a phenomenon known as stress shielding. Consequently, some fractures failed to fully heal properly when stabilized with stainless steel nails, or failed to heal quickly.

Delayed healing (delayed unions) and unhealed fractures (non-unions) can lead to significant quality of life problems for the patient. For example, patients who have not healed properly or steadily experience increased medical costs, reduced quality of life, and absenteeism from work. Therefore, any technology that accelerates bone healing and decreases the incidence of non-unions would be highly advantageous.

The late 1980s and early 1990s saw the development of intramedullary nails made from titanium. Titanium's more favorable mechanical properties improved fracture healing rates and reduced the incidence of delayed unions and non-unions.

Parallel to advancements in intramedullary nailing materials technology, research has highlighted the benefits of early motion on fracture healing. Despite substantial evidence from animal and human n studies demonstrating the advantages of controlled motion on fracture healing, there has been limited translation of this technology into clinical practice. This underscores the need for a medical device that enables a controlled amount of motion at the fracture site, facilitating optimal healing conditions.

The need for improvement in intramedullary nailing continues.

### SUMMARY

In an embodiment, an intramedullary nail includes a proximal body portion and a distal body portion coupled to each other. An inflection point is located along the proximal body portion, and an interlocking screw hole extends through the proximal body portion distal to the inflection point. The intramedullary nail has been found to unexpectedly reduce wear and provide a more durable solution to patients recovering from long bone fractures.

In an embodiment, an intramedullary nail includes a proximal body portion and a distal body portion coupled to each other and defining a length of the intramedullary nail extending between opposed proximal and distal ends of the intramedullary nail. An inflection point is located along the length, where the length is bifurcated into a proximal length proximal to the inflection point and a distal length distal to the inflection point. Proximal bores are located proximate the proximal end of the intramedullary nail. Distal bores are located proximate the distal end of the intramedullary nail. An intermediate bore is located proximate to the inflection point. The intermediate bore is located along the distal length.

In embodiments, the intermediate bore is located in the proximal body portion.

In embodiments, the proximal bores are clustered together in a proximal cluster, the distal bores are clustered together in a distal cluster, and the proximal cluster and the distal cluster are each spaced apart from the intermediate bore along the length of the intramedullary nail.

In embodiments, an upper portion of the proximal body portion and a lower portion of the proximal body portion are delineated by the inflection point. An upper proximal axis is defined about which the upper portion of the proximal body portion is coaxial. A lower proximal axis is defined about which the lower portion of the proximal body portion is coaxial and which is misaligned with the upper proximal axis.

In embodiments, the upper proximal axis and the lower proximal axis define and lie in a common plane extending through the proximal body portion, and the intermediate bore extends through the proximal body portion in a direction normal to the plane.

In embodiments, the proximal body portion and the distal body portion are coupled to each other for axial reciprocation. A biasing element is disposed between the proximal body portion and the distal body portion and is configured to bias the proximal body portion and the distal body portion apart from each other.

In embodiments, the biasing element is a spring constructed from a cobalt-chrome alloy.

In embodiments, an adjustment assembly is configured to limit a maximal amount of axial reciprocation of the proximal body portion and the distal body portion with respect to each other.

In an embodiment, an intramedullary nail includes a proximal body portion and a distal body portion coupled to each other and defining a length of the intramedullary nail extending between opposed proximal and distal ends of the intramedullary nail. The proximal body portion has an upper proximal axis and an opposed lower proximal axis which is misaligned with the upper proximal axis. The distal body portion has a distal axis. Proximal bores are located proximate the proximal end of the intramedullary nail, and the proximal bores are oriented normal to and extend through the upper proximal axis. Distal bores are located proximate the distal end of the intramedullary nail, and the distal bores are oriented normal to and extend through the distal axis. An intermediate bore is oriented normal to and extends through the lower proximal axis.

In embodiments, the intermediate bore is proximate to the upper proximal axis.

In embodiments, the intermediate bore is located proximate to an intersection of the upper proximal axis and the lower proximal axis.

In embodiments, the intermediate bore is located in the proximal body portion.

In embodiments, the proximal bores are clustered together in a proximal cluster, the distal bores are clustered together in a distal cluster, and the proximal cluster and the distal cluster are each spaced apart from the intermediate bore along the length of the intramedullary nail.

In embodiments, the upper proximal axis and the lower proximal axis define and lie in a common plane extending through the proximal body portion, and the intermediate bore extends through the proximal body portion in a direction normal to the plane.

In embodiments, the proximal body portion and the distal body portion are coupled to each other for axial reciprocation. A biasing element is disposed between the proximal body portion and the distal body portion and is configured to bias the proximal body portion and the distal body portion apart from each other. The biasing element includes a spring constructed from a cobalt-chrome alloy.

In an embodiment, an intramedullary nail includes a proximal body portion and a distal body portion coupled to each other and defining a length of the intramedullary nail extending between opposed proximal and distal ends of the intramedullary nail. The proximal body portion has a sidewall extending from a proximal body proximal end to an opposed proximal body distal end. The sidewall extends along an upper proximal axis from the proximal body proximal end and then bends at an inflection point, located between the proximal body proximal end and the proximal body distal end, thereafter extending from the inflection point along a lower proximal axis to the proximal body distal end. An intermediate bore is located between the inflection point and the proximal body distal end, and the intermediate bore extends through the sidewall of the proximal body portion.

In embodiments, the upper proximal axis and the lower proximal axis are misaligned with each other.

In embodiments, the upper proximal axis and the lower proximal axis define and lie in a common plane extending through the proximal body portion, and the intermediate bore extends through the proximal body portion in a direction normal to the plane.

In embodiments, a cluster of proximal bores is located proximate to the proximal end of the intramedullary nail. A cluster of distal bores is located proximate to the distal end of the intramedullary nail. The cluster of proximal bores and the cluster of distal bores are each spaced apart from the intermediate bore along the length of the intramedullary nail.

In embodiments, the proximal body portion and the distal body portion are coupled to each other for axial reciprocation. A biasing element is disposed between the proximal body portion and the distal body portion and is configured to bias the proximal body portion and the distal body portion apart from each other. The biasing element includes a spring constructed from a cobalt-chrome alloy.

The above provides the reader with a very brief summary of some embodiments described below. Simplifications and omissions are made, and the summary is not intended to limit or define in any way the disclosure. Rather, this brief summary merely introduces the reader to some aspects of some embodiments in preparation for the detailed description that follows.

### BRIEF DESCRIPTION OF THE DRAWINGS

Referring to the drawings:

FIGS. 1 and 2 are front and perspective views, respectively, of an improved dynamic axial nail for intramedullary treatment of long bone fractures (“nail”);

FIG. 3 is a side elevation view of the nail of FIG. 1;

FIG. 4 is an exploded side perspective view of a distal body portion of the nail of FIG. 1;

FIG. 5 is an exploded section view of the distal body portion, taken along the line 5-5 from FIG. 4;

FIG. 6 is a side perspective view of a proximal body portion of the nail of FIG. 1;

FIG. 7 is a section view of the proximal body portion, taken along the line 7-7 from FIG. 6;

FIG. 8 is a section view of the nail taken along the line 8-8 from FIG. 1; and

FIG. 9 is an enlarged section view of the nail taken along the line 8-8 from FIG. 1.

### DETAILED DESCRIPTION

Reference now is made to the drawings, in which the same reference characters are used throughout the different figures to designate the same elements. Briefly, the embodiments presented herein are preferred exemplary embodiments and are not intended to limit the scope, applicability, or configuration of all possible embodiments, but rather to provide an enabling description for all possible embodiments within the scope and spirit of the specification. Description of these preferred embodiments is generally made with the use of verbs such as “is” and “are” rather than “may,” “could,” “includes,” “comprises,” and the like, because the description is made with reference to the drawings presented. One having ordinary skill in the art will understand that changes may be made in the structure, arrangement, number, and function of elements and features without departing from the scope and spirit of the specification. Further, the description may omit certain information which is readily known to one having ordinary skill in the art to prevent crowding the description with detail which is not necessary for enablement. Indeed, the diction used herein is meant to be readable and informational rather than to delineate and limit the specification; therefore, the scope and spirit of the specification should not be limited by the following description and its language choices.

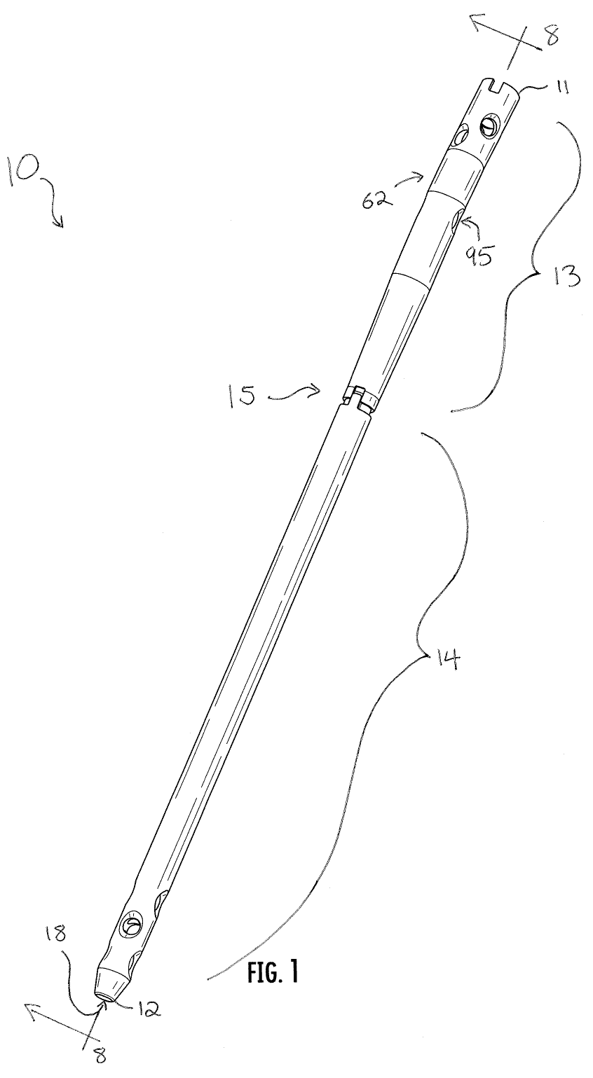

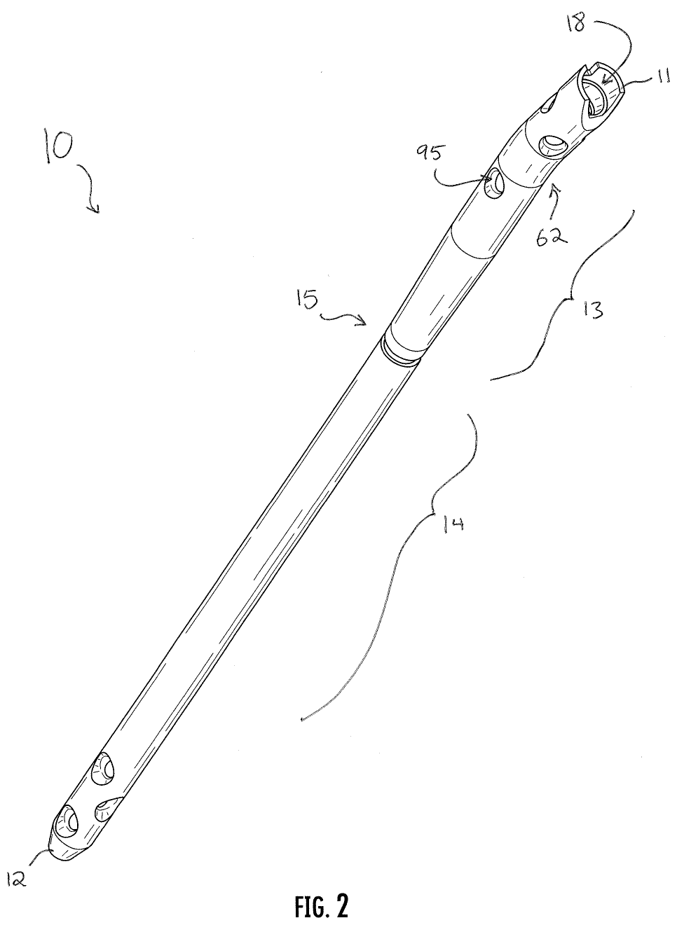

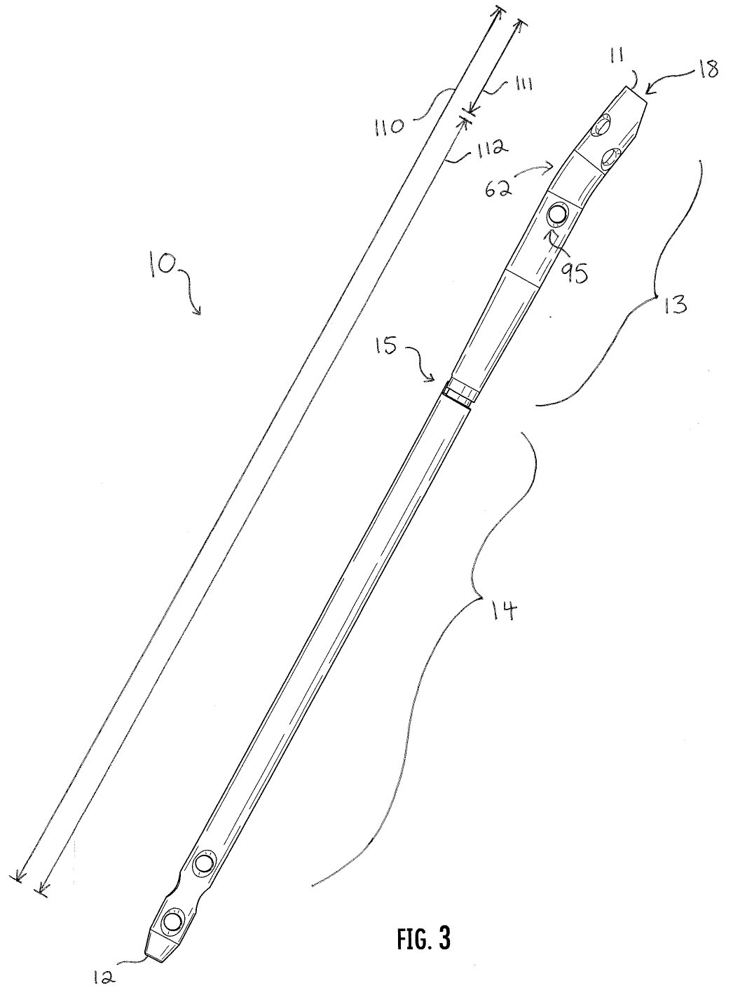

FIG. 1 is a “front” perspective elevation of an improved dynamic axial nail for intramedullary treatment of long bone fractures 10 (hereinafter, the “nail 10”). FIG. 2 is a “rear” perspective view of the nail 10, and FIG. 3 is a “side” elevation view of the nail 10. The nail 10 does not have a true front, rear, or side, but use of the terms is convenient and aids in the understanding of the nail 10, and so those terms, as well as terms like “top” and “bottom,” “upper” and “lower,” “above” and “below,” and the like may be used here with the understanding that such terms are used with their common meanings according to the orientation of the nail 10 on the page. Those terms are not meant to limit the nail 10 in any way.

The nail 10 is a multi-piece nail 10 having two major body portions which are mounted to each over for adjustable telescopic compression and axial reciprocation relative to each other. A medical worker installing the nail 10 in the leg of a patient is able to set a desired amount of movement or “travel” between the two major body portions. In other words, the medical worker can limit the maximal amount of axial reciprocation of the two major body portions with respect to each other. In some contexts, that may be only a millimeter or two of travel. In other situations, it may be more. In still other settings, the medical worker may set the nail 10 for zero travel, such that the two major body portions are fixed with respect to each other.

The nail 10 includes a proximal end 11, an opposed distal end 12, a proximal or proximal body portion 13 toward the proximal end 11, and an opposed distal or distal body portion 14, toward the distal end 12. The proximal and distal body portions 13 and 14 are coupled to each other at a juncture 15 formed by and between the proximal and distal body portions 13 and 14. In this embodiment, the juncture 15 preferably houses both a biasing element 16 biasing the proximal and distal body portions 13 and 14 apart from each other and an adjustment bolt 17 for regulating the amount of compression of the biasing element 16. The biasing element 16 and bolt 17 are visible in later drawings. A lumen 18 extends entirely through the nail 10, from the proximal end 11 to the distal end 12, through both the proximal and distal body portions 13 and 14.

Very generally, and again, without limitation, the proximal end 11 of the nail 10 is considered a “top” because it is oriented toward the top of the page in the drawings, and the distal end 12 of the nail 10 is considered a “bottom” because it is oriented toward the bottom of the page in the drawings. These terms are used convenience and readability only.

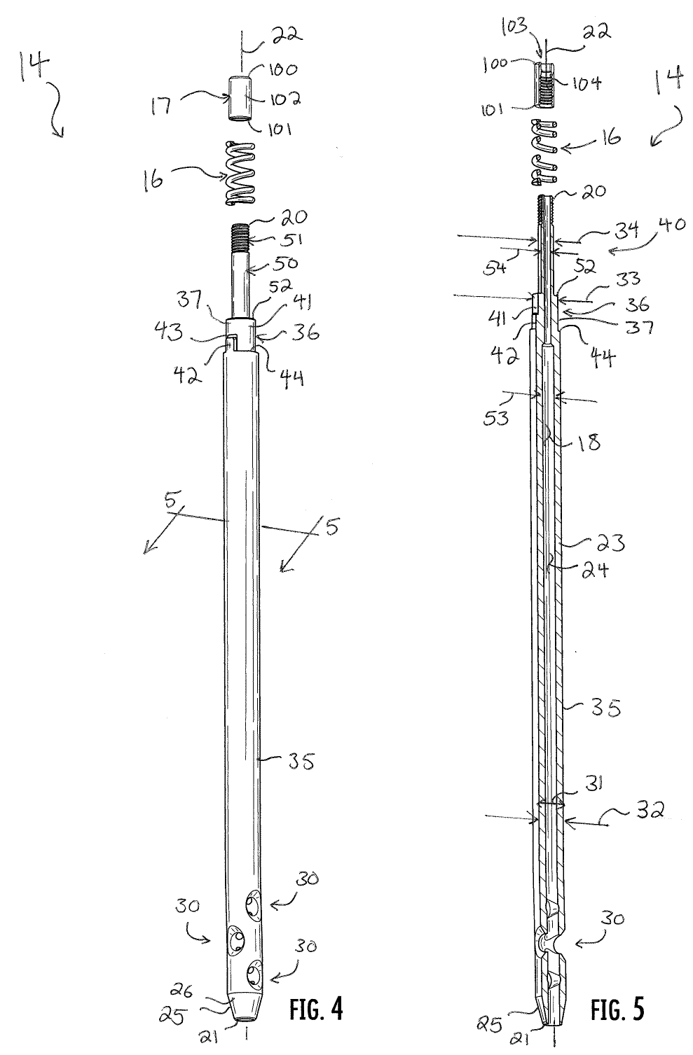

FIGS. 4 and 5 are exploded views of the distal body portion 14 of the nail 10. FIG. 5 is a section view taken along the line 5-5 in FIG. 4, bisecting the distal body portion 14. The distal body portion 14 of the nail 10 has a body extending from a distal body proximal end 20 (the “proximal end 20”) to an opposed distal body distal end 21 (the “distal end 21”) (identical to the distal end 12 of the entire nail 10 as an assembly). The distal body portion 14 is preferably generally linear between the proximal and distal ends 20 and 21, aligned along a first longitudinal or distal axis 22. The distal body portion 14 has a cylindrical sidewall 23 and a central open bore 24. The sidewall 23—and the distal body portion 14 itself—is coaxial to the distal axis 22 and nearly have rotational about the same, but with some exceptions noted below. The bore 24 is part of and defines the lumen 18 as it extends through the distal body portion 14 of the nail 10.

At the distal end 21, the distal body portion 14 terminates in a truncated conical tip 25. The tip 25 has a taper 26 which forms an approximately eleven-degree angle with the distal axis 22, though this angle is not critical and does vary in other embodiments depending on and as is suitable for the use context.

Just distal to or inboard of the tip 25 are three bores 30 extending transversely through the sidewall 23 of the distal body portion 14. These bores 30 are considered “distal bores” because they are located generally proximate to the distal end 12 of the nail 10. They are spaced relatively closely together along the distal axis 22, such that they define a cluster or “distal cluster,” again, because the cluster is proximate to the distal end 12 of the nail 10.

Each bore 30 extends through the sidewall 23 of the distal body portion 14. Each bore 30 is oriented normal to the distal axis 22, such that each has its own coaxial axis which is normal to the distal axis 22. Moreover, each of the axes of the bores 30—and so each of the bores 30—extends through the distal axis 22.

Each bore 30 is configured to receive a screw or other fastener for fixing the distal body portion 14 to a section of bone adjacent to the distal body portion 14. The bores 30 are longitudinally or axially spaced slightly apart on the distal body portion 14 and have different orientations through the distal body portion 14; in this embodiment, one of the bores 30 is circumferentially offset with respect to the other two. A medical worker is able to pass screws through one, two, or all three of the bores 30 as the fracture requires in the medical worker's judgment.

Three bores 30 are shown in this embodiment; other embodiments have a fewer or greater number of bores 30 spaced apart in different locations along the distal body portion 14. These drawings show a preferred embodiment, however.

Behind the tip 25, the sidewall 23 has a generally constant outer diameter 31 along the entirety of the distal body portion 14, except as specifically described below. Along most of the distal body portion 14, referred to herein as the shank 35, the outer diameter 31 has a first dimension 32.

The distal body portion 14 includes the tip 25, the long shank 35, and also a short stub 36 and a post 40 at the proximal end 20 of the distal body portion 14. The outer diameter 31 of the distal body portion 14 changes along these structures. The outer diameter 31 reduces or steps inward from the first dimension 32 of the shank 35 twice. This first dimension 32 characterizes the outer diameter 31 from just behind the tip 25 to the short stub 36 having a cylindrical bearing surface 37. The outer diameter 31 reduces at the stub 36. Then, proximally to the stub 36, the post 40 extends back to the proximal end 20 of the distal body portion 14, and the outer diameter 31 reduces from the stub 36 to the post 40.

The stub 36 is a short projection, integral to the shank 35, but extending proximally therefrom. The outer diameter 31 of the stub 36 is a second dimension 33, which is smaller than the first dimension 32 of the outer diameter 31 along the shank 35. The stub 36 has a smooth cylindrical outer surface 41 but for a tongue 42. The tongue 42 is contiguous to the outer surface 41 of the shank 35 and extends axially and proximally from that outer surface 41 approximately halfway along the axial length of the stub 36. In some embodiments, such as those shown in these drawings, a small channel 43 extending radially into the stub 36 borders the tongue 42.

There is a distally-facing shoulder 44 between the first dimension 32 of the shank 35 and the second dimension 33 of the stub 36. The shoulder 44 is an annular ring severed or interrupted by the tongue 42. The shoulder 44 steps down from the shank 35 to the stub 36. The stub 36 is a mounting location for the proximal body portion 13, and the outer surface 37 of the stub 36 is a bearing surface for the proximal body portion 13. The proximal body portion 13 rides on and slides over the bearing surface 37. The shoulder 44 is an interference face directed proximally or toward the proximal end 20. The shoulder 44 limits movement of the proximal body portion 13 distally with respect to the distal body portion 14.

The post 40 extends distally from the stub 36, integral to the stub 36 and the shank 35. The outer diameter 31 of the post 40 is a third dimension 34, which is smaller than the second dimension 33 of the outer surface 37 along the stub 36. The post 40 has a smooth portion 50 and a threaded portion 51. The threaded portion 51 is proximate to the proximal end 20 of the distal body portion 14. The smooth portion 50 extends between the threaded portion 51 and the stub 36. The outer surface of the smooth portion 50 is preferably smooth and uninterrupted by discontinuities, projections, channels, and the like. The outer surface e of the threaded portion 51 is formed with outwardly-directed threads.

There is a distally-facing shoulder 52 between the second dimension 33 of the stub 36 and the third dimension 34 of the post 40. The shoulder 52 is a continuous annular ring without interruption. Like the shoulder 44, this shoulder 52 is also an interference face. The post 40 is a mounting location for the biasing element 16, for the proximal body portion 13 of the nail 10, and for the adjustment bolt 17, as explained below.

The bore 24 extends entirely through the distal body portion 14. It has two inner diameters: a first inner diameter 53 and a second inner diameter 54. The second inner diameter 54 is smaller than the first inner diameter 53. The first inner diameter 53 defines the internal dimension of the bore 24 from the distal end 21 of the distal body portion 14 to a location, distal to the shoulder 44, that is formed between the shank 35 and the stub 36. There, the bore 24 reduces to the second inner diameter 54. The second inner diameter 54 is constant from that location to the proximal end 20 of the distal body portion 14.

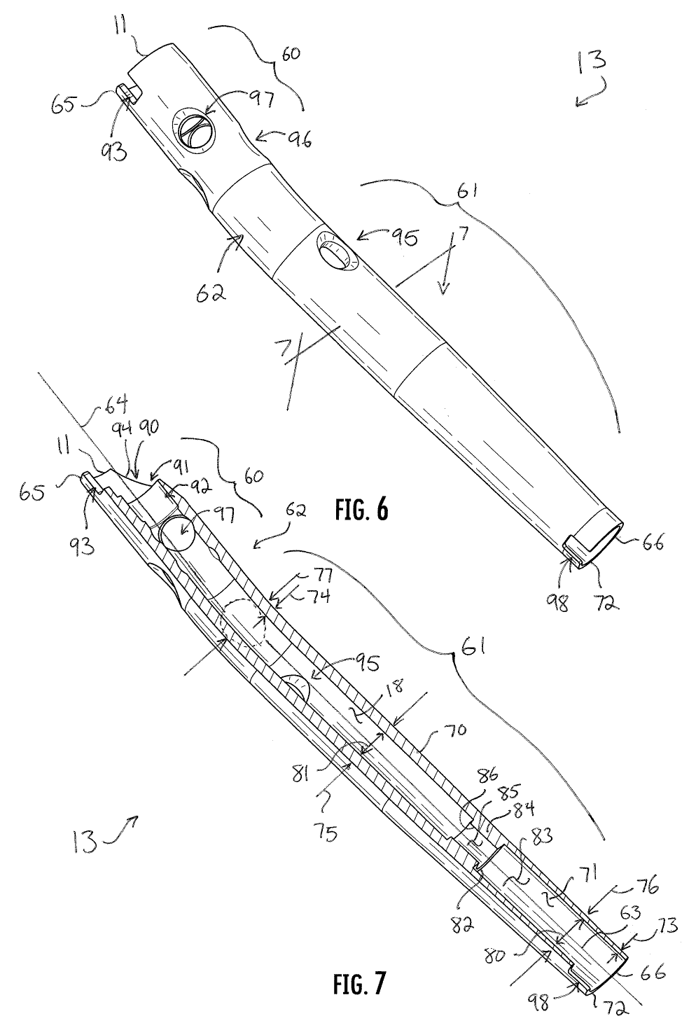

The proximal body portion 13 of the nail 10 fits over the distal body portion 14 of the nail 10. They are coupled to each other for axial reciprocation. The biasing element 16 is disposed between the body portions 13 and 14 to bias them apart from each other. FIGS. 6 and 7 show the proximal body portion 13 in isolation in perspective views; FIG. 6 is an exterior view, and FIG. 7 is a section view taken along the line 7-7 in FIG. 6.

The proximal body portion 13 is a shank extending from a proximal body proximal end 65 (the “proximal end 65”) (identical to the proximal end of the nail 10) to an opposed proximal body distal end 66 (the “distal end 66”). The proximal body portion 13 has an upper portion 60 and an opposed lower portion 61 which are formed as a single monolithic, integral pieces but are oriented non-linearly or obliquely with respect to each other. The upper and lower portions 60 and 61 meet at and are delineated by an inflection point 62 where the proximal body portion 13 is bent. The inflection point 62 is identified generally by a circle drawn in broken line in FIGS. 6 and 9.

The inflection point 62 not only delineates the upper and lower portions 60 and 61 of the proximal body portion 13 but also bifurcates the length of the nail 10. Specifically, the nail 10 defines an overall longitudinal length 110 extending from the proximal end 11 to the opposed distal end 12, as shown in FIG. 3. The segment of this length 110 extending from the proximal end 11 to the inflection point 62 is referred to as the proximal length 111. This proximal length 111 is proximate to the proximal end 11 and is rigid and unchanging. In a preferred embodiment, the proximal length 111 is approximately 31.4 millimeters, in comparison to the length of the proximal body portion 13 of approximately 104.9 millimeters from its proximal end 65 to its distal end 66. The segment of the length 110 extending from the inflection point 62 to the distal end 12 of the nail is referred to as the distal length 112. This distal length 112 includes the lower portion 61 of the proximal body portion 13, the entire distal body portion 14, and the juncture 15 formed between the proximal and distal body portions 13 and 14. It is distal to the inflection point 62. Because the proximal and distal body portions 13 and 14 are designed to allow reciprocation, this distal length 112 can potentially change with time, either rapidly and cyclically, or slowly over time. The length 110 can similarly change.

The lower portion 61 has a lower proximal axis 63, and the upper portion 60 has an upper proximal axis 64. The two axes 63 and 64 are misaligned with each other at an angle θ of approximately eleven degrees (best seen in FIG. 9), though this angle is not critical and does vary in other embodiments depending on and as is suitable for the use context.

The proximal body portion 13 has a cylindrical sidewall 70 which is integral and contiguous across the upper and lower portions 60 and 61 of the proximal body portion 13. The sidewall 70 extends along the upper proximal axis 64 from the proximal end 65 to the inflection point 62, where the sidewall 70 bends. The sidewall 70 then extends from the inflection point 62 along the lower proximal axis 63 to the distal end 66 of the proximal body portion 13. The sidewall 70 is coaxial to the upper and lower proximal axes 64 and 63 along the upper and lower portions 60 and 61, respectively. In other words, the upper portion 60 of the proximal body portion 13 is coaxial about the upper proximal axis 64, and the lower portion 61 of the proximal body portion 13 is coaxial about the lower proximal axis 63. These two axes 63 and 64 are misaligned with respect to each other.

The sidewall 70 of the proximal body portion 13 bounds a defines a central open bore 71 extending entirely through the proximal body portion 13 from the proximal end 65 to the distal end 66. The bore 71 is part of and defines the lumen 18 as it extends through the proximal body portion 13 of the nail 10. When the proximal and distal body portions 13 and 14 of the nail 10 are coupled at the juncture 15, the bores 24 and 71 of the distal and proximal body portions 14 and 13 are in open communication with each other and form the entire lumen 18.

At its distal end 66, the proximal body portion 13 terminates in an interference face 72. The interference face 72 is annular and thin. The interference face 72 is the distal termination of the sidewall 70 of the proximal body portion 13. Near the distal end 66, the thickness of the sidewall 70 has a minimum thickness dimension 73. While proximate the distal end 66, the sidewall 70 is thin, as it extends proximally it increases in thickness gradually to a second or maximum thickness dimension 74. That maximum thickness dimension 74 is located at the inflection point 62 in the proximal body portion 13, generally intermediate the proximal and distal ends 65 and 66 of the proximal body portion 13. The thickness of the sidewall 70 is then constant from the inflection point 62 to the proximal end 65 of the proximal body portion 13, except as described below.

The proximal body portion 13 has an outer diameter 75 that also increases, generally, from the distal end 66 to the proximal end 65. At the distal end 66, the outer diameter 75 has a minimal first dimension 76. The outer diameter 75 then increases gradually until it reaches a maximal second dimension 77 at the inflection point 62. Between the inflection point 62 and the proximal end 65, the outer diameter 75 remains constant.

Opposite the outer diameter 75, the bore 71 of the proximal body portion 13 extends entirely therethrough in an axial direction. Proximate the distal end 66, the bore 71 has a first inner diameter 80. This first inner diameter 80 extends constantly from the distal end 66 to an interior endwall 82 within the proximal body portion 13. The endwall 82 faces the distal end 66 and is annular. It defines an interference face for the biasing element 16 compressed between it and the shoulder 52 of the distal body portion 14. Together with the sidewall 70, the distal end 66 and the endwall 82 bound and define a holding space 83 for the biasing element 16. The holding space 83 is part of the juncture 15 formed by and between the proximal and distal body portions 13 and 14. Briefly, as used herein, the term “biasing element” refers broadly to any component or combination of components that produce a restoring force or displacement pressure between two other components. Such biasing elements may act mechanically, elastically, hydraulically, pneumatically, magnetically, thermally, or through other physical principles.

The endwall 82 is a distal face of an internal flange 84. The flange 84 extends radially inward from the sidewall 70, defining an aperture 85 through which the post 40 of the distal body portion 14 extends when the proximal and distal body portions 13 and 14 are coupled. The flange 84 is part of the juncture 15 formed by and between the proximal and distal body portions 13 and 14. The flange 84 projects inwardly, terminating with an inner surface 86 having an inner diameter less than the inner diameter around the flange 84. Proximally from the flange 84, the bore 71 has a second inner diameter 81. This second inner diameter 81 is less than the first inner diameter 80 and is constant through the remainder of the lower portion 61 of the proximal body portion 13 to the inflection point 62. At the inflection point 62, the bore 71 continues toward the proximal end 65 with the constant second inner diameter 81 until a socket 90 at the proximal end 65.

The socket 90 is in open communication with, and forms part of, the bore 71. The socket 90 includes a cylindrically-shaped first recess 91 extending into the upper portion from the proximal end 65. It has a depth and an inner diameter. The socket 90 is deeper than just the first recess 91, however: a second recess 92 extends distally beyond the first recess 91 to a depth roughly equivalent to the depth of the first recess 91 and with an inner diameter that is larger than the inner diameter of the first recess 91. In these drawings, the socket 90 has a smooth inner surface across the first and second recesses 91 and 92. However, generally the socket 90 has a threaded inner surface so that a bolt may be threadably engaged with the socket 90.

A small rectangular notch 93 is formed into the sidewall 70 of the proximal body portion 13 at the first recess 91. The notch 93 passes entirely through one side of the sidewall 70 and is thus in communication with the socket 90. The notch 93 extends axially from the proximal end 65 just longer than halfway along the first recess 91.

Opposite the notch 93 is a bevel 94. The bevel 94 extends from the proximal end 65, inboard of the sidewall 70, and cuts diagonally through the sidewall 70, such that there is no sidewall 70 on one side of the first recess 91, though the second recess 92 is encircled by the sidewall 70.

At the other end of the proximal body portion 13, at the distal end 66, and registered with the notch 93, is another notch 98. This notch 98 passes entirely through the sidewall 70 of the proximal body portion 13 at the holding space 83 and is thus in communication with the holding space 83. The notch 98 extends proximally from the distal end 66. It is sized and shaped to closely receive the tongue 42 on the distal body portion 14.

Several bores 95, 96, and 97 are formed through the sidewall 70. An interlocking screw hole, or intermediate bore 95, extends transversely through the sidewall 70 of the proximal body portion 13 distal to the inflection point 62, between the inflection point 62 and the distal end 66. The upper bores 96 and 97 extend transversely through the sidewall 70 of the proximal body portion 13 proximal to the inflection point 62, between the inflection point 62 and the proximal end 11.

The intermediate bore 95 extends through the proximal body portion 13 in a direction normal to the lower proximal axis 63, such that the bore 95 has its own coaxial axis which is normal to the lower proximal axis 63. Moreover, the axis of the bore 95 extends through the lower proximal axis 63. In contrast, the upper bores 96 and 97 extend transversely through the sidewall 70 between the inflection point 62 and the proximal end 65. The upper bores 96 and 97 extend through the upper portion 14 preferably normal to and through the upper proximal axis 64, such that those bores 96 and 97 have their own coaxial axes which are normal to the upper proximal axis 64, and the axes of the bores 96 and 97 extend through the upper proximal axis 64.

In contrast to the intermediate bore 95, the upper bores 96 and 96 are considered “proximal bores” because they are located generally proximate to the proximal end 11 of the nail 10. They are spaced relatively closely together along the upper proximal axis 64, such that they define a cluster or “proximal cluster,” again, because the cluster is proximate to the proximal end 11 of the nail 10. The proximal cluster of bores 96 and 97, and the distal cluster of bores 30 are both spaced apart from the intermediate bore 95 along the length 110 of the nail 10.

Each of the bores 95-97 is configured to receive a screw or other fastener for fixing the proximal body portion 13 to a section of bone adjacent to the proximal body portion 13. The bores 96 and 97 are longitudinally spaced apart on the proximal body portion 13 and have different orientations through the proximal body portion 13 from each other and from the intermediate bore 95, thereby allowing a medical worker to pass screws through one, two, or all of the bores 95-97 as the fracture requires in the medical worker's judgment. If the medical worker places only one screw, then the medical worker places the one screw through the intermediate bore 95 below the inflection point 62. And if the medical worker places multiple screws, then the medical worker preferably places the first screw through the intermediate bore 95. The bores 96 and 97 are circumferentially spaced apart in this embodiment. Two upper bores 96 and 96 are shown in this embodiment; other embodiments have a fewer or greater number of bores spaced apart in different locations along the proximal body portion 13. These drawings show a preferred embodiment, however.

The intermediate bore 95 is proximate to the inflection point 62, just distal thereto. The intermediate bore 95 is located along the distal length 112, but in the lower portion 61 of the proximal body portion 13. The intermediate bore 95 is located approximately 43.0 millimeters from the proximal end 11 and is thus approximately 11.6 millimeters distal to the inflection point 62. Moreover, the intermediate bore 95 is proximate to the upper proximal axis 64 but does not actually align with or extend through the upper proximal axis 64. Rather, the intermediate bore 95 is proximate to an intersection 99 of the upper proximal axis 64 and the lower proximal axis 63, located distally with respect to that intersection 99. This intersection 99 is shown in broken line in FIG. 9 within the inflection point 62.

Still referring to FIG. 9, the upper and lower proximal axes 63 and 64 are shown extending through the proximal body portion 13. These two axes 63 and 64 are coplanar; they define and lie in a common plane which extends through the proximal body portion 13. That common plane also includes the section line 9-9 from FIG. 1 which yields the section view of FIG. 9. As such, the common plane is parallel to the page on which the drawing of FIG. 9 appears, and so cannot be shown or marked in the drawing. The intermediate bore 95 extends through the lower portion 61 of the proximal body portion 13 in a direction normal to that plane. In other words, the coaxial axis of the intermediate bore 95 is registered normal with that plane. As further shown in FIG. 9, the intermediate bore 95 is registered with the distal axis 22. The distal axis 22 and the lower proximal axis 63 are registered with each other, and so the intermediate bore 95 extends through and is normal to the distal axis 22.

In operation, the nail 10 is used to dynamically fix two or more pieces of fractured bone together. For example, in tibial shaft fractures—the most common long bone fracture in the United State—a health worker reams the intramedullary canal to create an internal bore in preparation for application of the nail 10. The nail 10 is preferably pre-assembled, and the health worker only needs to set the desired axial reciprocal movement of the proximal and distal body portions 13 and 14. The health worker adjusts the adjustment bolt 17 to do this.

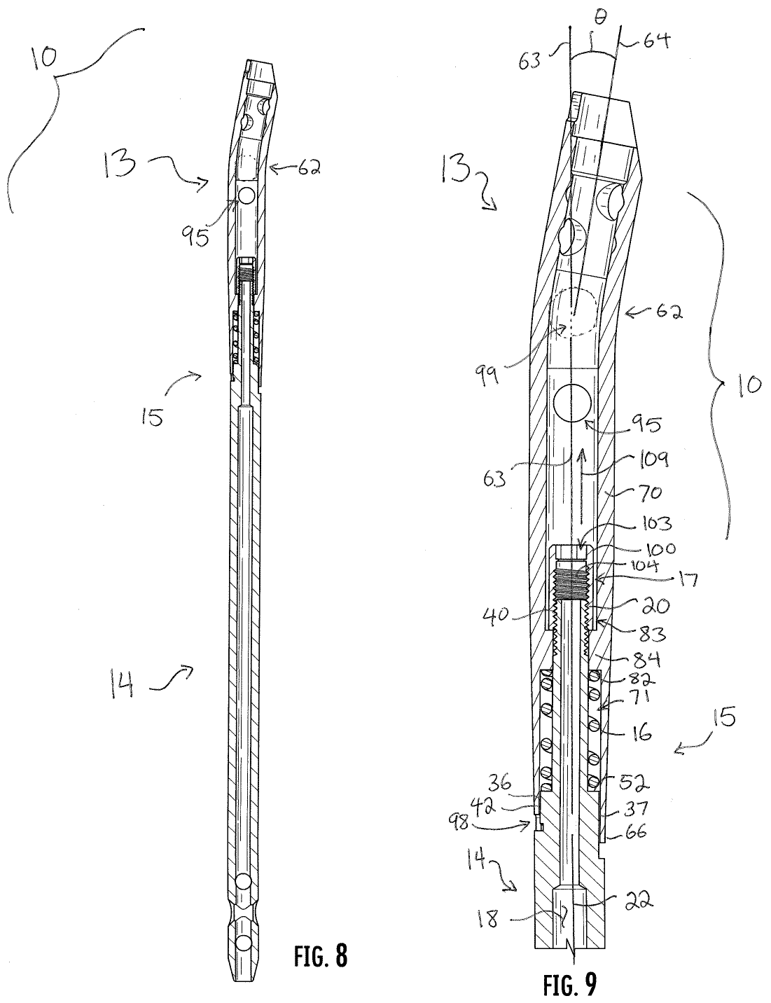

FIGS. 8 and 9 show the juncture 15. The distal end 66 of the proximal body portion 13 fits over the proximal end 20 of the distal body portion 14. The holding space 83, the internal flange 84, and the bore 71 of the proximal body portion 13 receive the stub 36 and post 40 of the distal body portion 14. The tongue 42 is received closely in the notch 98, thereby preventing relative rotational movement of the proximal and distal body portions 13 and 14. The inner surface of the sidewall 70 of the proximal body portion 13, proximate the distal end 66, is received in contact against the outer bearing surface 37 of the stub 36.

The holding space 83 is then filled by the post 40 and the biasing element 16. In this embodiment, the biasing element is a helical compression spring 16 encircling the post 40. The spring 16 is captured radially between the post 40 and the sidewall 70. The spring 16 is captured axially between the shoulder 52 of the distal body portion 14 and the endwall 82 of the internal flange 84 on the proximal body portion 13. The inner surface 86 of the internal flange 84 is in contact with the outer surfaces of the smooth and threaded portions 50 and 51.

The spring 16 biases the proximal body portion 13 away from the distal body portion 14 along an axial direction shown by the arrowed line 109 in FIG. 9. The spring 16 pushes against the shoulder 52 and the endwall 82, urging those two faces apart from each other so as to separate the proximal and distal body portions 13 and 14. A helical compression spring 16 is shown in the embodiment of these drawings. While a helical compression spring is shown and described in the illustrated embodiment, the biasing element may instead include any mechanical, elastomeric, fluidic, or electromechanical structure capable of generating a restoring force between the proximal and distal body portions. Suitable biasing elements include, but are not limited to, coil springs, leaf springs, Belleville washers, elastomeric members such as rubber or silicone boots, closed-cell foams, bladder-type springs filled with gas or fluid, hydraulic or pneumatic pistons, solenoids, magnetic or electromagnetic repulsion systems, and shape-memory alloys that produce a restoring force when activated. Combinations of such elements may also be used. The specific choice of biasing element depends on desired force characteristics, fatigue tolerance, sterilization compatibility, and other medical or engineering factors. The spring 16 is shown here for simplicity. Where an embodiment of the nail 10 does employ a helical compressional spring 16, the spring 16 is preferably constructed from stainless steel or cobalt-chrome alloy. Embodiments constructed from stainless steel preferably are SS316L, and embodiments which are cobalt-chrome alloys are preferably FWM 1058 or 35N LT. These materials are identified as preferred embodiments and are not intended to limit the scope of the disclosure of the spring 16. Other spring materials are suitable, such as titanium alloy, polyethylene, etc.

The adjustment bolt 17 limits the separation of the proximal and distal body portions 13 and 14 to an adjustable maximum. The adjustment bolt 17 has a proximal end 100 and an opposed distal end 101. A hollow, cylindrical sidewall 102 extends between the proximal and distal ends 100 and 101. At the proximal end 100, the sidewall 102 has an internal hex socket 103 such that an allen or hex tool can be inserted therein to turn the adjustment bolt 17. An inner surface 104 of the sidewall 102 is formed with threads complemental to the threads on the threaded portion 51 of the post 40. As such, the adjustment bolt 17 is threadably engaged to the post 40 and can be rotated in one direction and the other to selectively advance or retract the adjustment bolt 17 over the post 40 in an axial direction, thereby decreasing or increasing the axial length of the holding space 83, respectively.

When the health worker turns the adjustment bolt 17 in one direction, the adjustment bolt 17 moves closer to the stub 36. The distal end 101 of the adjustment bolt 17 pushes the internal flange 84 axially distally, thereby decreasing the axial length of the holding space 83. In contrast, when the health worker turns the adjustment bolt 17 in an opposite direction, the adjustment bolt 17 moves further from the stub 36. The distal end 101 of the adjustment bolt 17 backs off the internal flange 84. The spring 16 then pushes the internal flange 84 proximally in confrontation with the adjustment bolt 17. These structures—the adjustment bolt 17, the post 40, the flange 84, the spring 16 and its interaction with the endwall 82 and shoulder 52—define an adjustment assembly allowing the health worker to adjust the nail 10.

In this way, the health worker can set the desired amount of axial reciprocal movement or play in the nail 10. If the health worker believes there should be no axial movement in the nail 10—that the nail 10 should be essentially static—then he turns the adjustment bolt until the distal end 66 of the proximal body portion 13 makes contact with the shoulder 44. The shoulder 44 prevents axial movement in a distal direction of the proximal body portion 13 over the distal body portion 14, and the adjustment bolt 17 prevents axial movement in a proximal direction of the proximal body portion 13 over the distal body portion 14. If the health worker needs to allow some axial play, he backs the adjustment bolt 17 off from the position a turn or two. The spring 16 pushes the internal flange 84 away from the distal body portion 14, thereby separating the proximal and distal body portions 13 and 14. However, the inner surface of the sidewall 70 at the distal end 66 of the proximal body portion 13 slides on the outer bearing surface 37, and the proximal body portion 13 can slide distally over the distal body portion 14 just slightly, thereby allowing limited amount of axial movement at the fracture to promote the healing process.

Once the limit of axial play has been set for the nail 10, a guidewire is passed into the bore reamed through intramedullary canal, and the nail 10 is then threaded onto the guidewire and applied to the intramedullary canal bore. Once the nail 10 is properly applied, it can be secured to the bone. Fixation or locking screws are placed through some or all of the bores 30, 95, 96, and 97 to ensure that the nail forms a static construct with rotational stability within the bone. The health worker applies a screw through the bone and the interlocking screw hole, or intermediate bore 95, first. If the health worker applies no other screws, this ensures that loading forces are applied to the bone along the centerline or distal axis 22 of the nail 10 and not off-center. If the health worker applies other screws, this first screw through the intermediate bore 95 still ensures that centered loading.

After recovery from surgery, the patient can now walk on the leg in a way that effectively promotes fracture repair. If the health worker has disabled axial play by bottoming out the adjustment bolt 17 so that the proximal and distal body portions 13 and 14 cannot move axially with respect to each other, then the nail 10 will be rigid and will not compress or decompress. If, on the other hand, the health worker has enabled axial play in the nail 10, then as the patient walks and puts weight on the leg, the nail 10 will compress and decompress axially at the juncture 15. The nail 10 is capable of behaving this way for several hundred thousand to millions of cycles. In some embodiments, after several hundred thousand cycles, the juncture 15 may begin to close. In such embodiments, the adjustment bolt 17 moves progressively closer to the stub 36 until it stops moving entirely. At this point, such embodiments of the nail 10 becomes rigid and no longer axially compresses or decompresses.

A preferred embodiment is fully and clearly described above so as to enable one having skill in the art to understand, make, and use the same. Those skilled in the art will recognize that modifications may be made to the description above without departing from the spirit of the specification, and that some embodiments include only those elements and features described, or a subset thereof. To the extent that modifications do not depart from the spirit of the specification, they are intended to be included within the scope thereof.

What is claimed is:

1. An intramedullary nail comprising:
a proximal body portion and a distal body portion coupled to each other, defining a length of the intramedullary nail extending between opposed proximal and distal ends of the intramedullary nail;
an inflection point along the length, wherein the length is bifurcated into a proximal length proximal to the inflection point and a distal length distal to the inflection point;
proximal bores proximate the proximal end of the intramedullary nail;
distal bores proximate the distal end of the intramedullary nail; and
an intermediate bore proximate to the inflection point;
wherein the intermediate bore is located along the distal length.

2. The intramedullary nail of claim 1, wherein the intermediate bore is located in the proximal body portion.

3. The intramedullary nail of claim 1, wherein the proximal bores are clustered together in a proximal cluster, the distal bores are clustered together in a distal cluster, and the proximal cluster and the distal cluster are each spaced apart from the intermediate bore along the length of the intramedullary nail.

4. The intramedullary nail of claim 1, further comprising:
an upper portion of the proximal body portion and a lower portion of the proximal body portion, wherein the inflection point delineates the upper portion and the lower portion;
an upper proximal axis about which the upper portion of the proximal body portion is coaxial; and
a lower proximal axis about which the lower portion of the proximal body portion is coaxial and which is misaligned with the upper proximal axis.

5. The intramedullary nail of claim 4, wherein the upper proximal axis and the lower proximal axis define and lie in a common plane extending through the proximal body portion, and the intermediate bore extends through the proximal body portion in a direction normal to the plane.

6. The intramedullary nail of claim 1, wherein:
the proximal body portion and the distal body portion are coupled to each other for axial reciprocation; and
a biasing element is disposed between the proximal body portion and the distal body portion and is configured to bias the proximal body portion and the distal body portion apart from each other.

7. The intramedullary nail of claim 6, wherein the biasing element is a spring.

8. The intramedullary nail of claim 6, further comprising an adjustment assembly configured to limit a maximal amount of axial reciprocation of the proximal body portion and the distal body portion with respect to each other.

9. An intramedullary nail comprising:
a proximal body portion and a distal body portion coupled to each other, defining a length of the intramedullary nail extending between opposed proximal and distal ends of the intramedullary nail;
the proximal body portion has an upper proximal axis and an opposed lower proximal axis which is misaligned with the upper proximal axis;
the distal body portion has a distal axis;
proximal bores proximate the proximal end of the intramedullary nail, the proximal bores oriented normal to and extending through the upper proximal axis;
distal bores proximate the distal end of the intramedullary nail, the distal bores oriented normal to and extending through the distal axis; and
an intermediate bore oriented normal to and extending through the lower proximal axis.

10. The intramedullary nail of claim 9, wherein the intermediate bore is proximate to the upper proximal axis.

11. The intramedullary nail of claim 9, wherein the intermediate bore is located proximate to an intersection of the upper proximal axis and the lower proximal axis.

12. The intramedullary nail of claim 9, wherein the intermediate bore is located in the proximal body portion.

13. The intramedullary nail of claim 9, wherein the proximal bores are clustered together in a proximal cluster, the distal bores are clustered together in a distal cluster, and the proximal cluster and the distal cluster are each spaced apart from the intermediate bore along the length of the intramedullary nail.

14. The intramedullary nail of claim 9, wherein:
the upper proximal axis and the lower proximal axis define and lie in a common plane extending through the proximal body portion; and
the intermediate bore extends through the proximal body portion in a direction normal to the plane.

15. The intramedullary nail of claim 9, wherein:
the proximal body portion and the distal body portion are coupled to each other for axial reciprocation;
a biasing element is disposed between the proximal body portion and the distal body portion and is configured to bias the proximal body portion and the distal body portion apart from each other; and
the biasing element comprises a spring.

16. An intramedullary nail comprising:
a proximal body portion and a distal body portion coupled to each other, defining a length of the intramedullary nail extending between opposed proximal and distal ends of the intramedullary nail;
the proximal body portion having a sidewall extending from a proximal body proximal end to an opposed proximal body distal end;
the sidewall extends along an upper proximal axis from the proximal body proximal end and then bends at an inflection point, located between the proximal body proximal end and the proximal body distal end, thereafter extending from the inflection point along a lower proximal axis to the proximal body distal end; and
an intermediate bore located between the inflection point and the proximal body distal end, the intermediate bore extending through the sidewall of the proximal body portion.

17. The intramedullary nail of claim 16, wherein the upper proximal axis and the lower proximal axis are misaligned with each other.

18. The intramedullary nail of claim 17, wherein:
the upper proximal axis and the lower proximal axis define and lie in a common plane extending through the proximal body portion; and
the intermediate bore extends through the proximal body portion in a direction normal to the plane.

19. The intramedullary nail of claim 16, further comprising;
a cluster of proximal bores proximate to the proximal end of the intramedullary nail;
a cluster of distal bores proximate to the distal end of the intramedullary nail; and
the cluster of proximal bores and the cluster of distal bores are each spaced apart from the intermediate bore along the length of the intramedullary nail.

20. The intramedullary nail of claim 16, wherein:
the proximal body portion and the distal body portion are coupled to each other for axial reciprocation;
a biasing element is disposed between the proximal body portion and the distal body portion and is configured to bias the proximal body portion and the distal body portion apart from each other; and
the biasing element comprises a spring.
